(19) United States
(12) Patent Application Publication
(10) Pub. No.: US 2012/0029646 A1
(43) Pub. Date: Feb. 2, 2012

(54) Mandible reconstruction system and method

(76) Inventor: Rui P. Fernandes, Jacksonville, FL (US)

(21) Appl. No.: 13/135,132

(22) Filed: Jun. 27, 2011

Related U.S. Application Data
(60) Provisional application No. 61/398,442, filed on Jun. 25, 2010.

(51) Int. Cl. A61F 2/28

(52) U.S. Cl. 623/17.17

(57) ABSTRACT

A system, method and devices for mandible reconstruction wherein a replacement mandible, either full or partial, is formed by cutting and reconnecting a donor bone, the devices comprising guide or template members that provide the correct angles needed for cutting the donor bone in order to replicate the configuration of the mandible being reconstructed. The system comprises a plurality of wedge-shaped members.

This application claims the benefit of U.S. Provisional Patent Application Ser. No. 61/398,442, filed Jun. 25, 2010.

### BACKGROUND OF THE INVENTION

This invention relates generally to the field of devices used in mandible reconstruction and their method of use, and more particularly to tool sets or instrumentation used in the creation of a replacement mandible formed from a donor bone. Even more particularly, the invention relates to template-type or guide devices and their use in a reconstruction methodology, the devices being guides for properly cutting the donor bone such that proper angles are easily obtained in donor bone segments used in the reconstruction of the mandible.

Disease or trauma may necessitate the removal and replacement of portions or all of a human mandible, i.e., the lower jaw. The human mandible is a generally U-shaped bone with the open portion extending rearward or posteriorly on the skull. The front or anterior portion of the mandible comprises the closed portion of the “U” and is referred to as the parasymphysis. Extending posteriorly from each end of the parasymphysis is a portion referred to as the mandible body, which comprises the anterior portion of the legs of the “U”, and extending in an upwardly angled manner from the posterior end of each mandible body is a ramus, comprising the posterior portion of the legs of the “U”. Each of the free ends of the rami ends in a condyle, a smooth rounded projection for articulation of the mandible with the skull at the tempromandibular joints.

In one reconstruction technique, a replacement mandible or a portion of a mandible is created by joining segments of donor bone, such as for example a fibula. Because the donor bone is substantially straight, whereas the mandible possesses angles and curved portions, the fibula is cut into segments with the ends of the segments angled such that when the segments are connected in end-to-end manner, the resulting construct has a configuration approximating the angles and curvature of the mandible. In this manner for example the combination of a ramus and mandible body, the combination of a mandible body and parasymphysis, the combination of a ramus, mandible body and parasymphysis, or an entire mandible can be recreated from properly cut and re-connected segments of the donor bone.

In a most basic technique, the surgeon determines the required dimensions and end angles of the donor bone segments by visual approximation. With this method, achieving the proper angle so that the configuration of the joined bone segments closely approximates the portion of the mandible being replaced is difficult, and often results in the need for multiple cutting and shaving operations until the proper end angles are achieved. To address the inaccuracy problem it is known to utilize a fibular cutting guide that consists of an elongated body, generally C-shaped in transverse cross-section, the body having a V-shaped cut-out positioned in the interior. The surgeon places the cutting guide onto the fibula and then uses the edges of the cut-out as a cutting guide such that a predetermined angle can be cut. While this technique and tool increases accuracy, the devices are relatively bulky, cannot be used with short bone segments, and providing multiple cutting guides with different angles is unwieldy and expensive.

It is an object of this invention to provide a solution to the accuracy problem encountered in the cutting of donor bone segments utilized for construction of a replacement mandible or mandible portion, the solution comprising a technique, system and devices that are relatively simple in construct, easy and low cost to manufacture, readily storable in small volume, and which enable the surgeon to accurately cut multiple angles on the ends of the donor bone that will result in the production of an accurate mandible reconstruction.

### SUMMARY OF THE INVENTION

A system, method and set of devices for mandible reconstruction wherein a replacement mandible, either full or partial, is formed by cutting and reconnecting a donor bone, such as a fibula, the devices comprising wedge-shaped or triangular guide or template members, the distal angles of which provide the correct angles needed for cutting the donor bone in order to replicate the configuration of the mandible or mandible portion being reconstructed. The system or set of devices may comprise a relatively small number of guide members chosen to provide the angles necessary for reproducing a typical mandible, or may comprise a larger number of guide members such that the surgeon can select the precise angles needed for a particular mandible reconstruction.

To create a replacement construct from a linear donor bone for a curved or angled portion of the mandible in order to reconstruct, for example, the combination of the parasymthysis and the mandible body or the combination of the ramus and the mandible body, a guide member of proper angle is chosen and used by the surgeon as a guide for the cutting blade, or as a means for marking or inscribing the proper cutting lines onto the donor bone. The surgeon then cuts the linear donor bone using a saw or similar device. The two angled ends of the bone segments are now joined in known manner, such as by bonding, such that the resulting construct will have the necessary angle to approximately reproduce the portion of the mandible being replaced. To create the proper end angles to reconstruct the portion of the mandible comprising the ramus and mandible body, after creating the proper main angle, the surgeon may utilize a second guide member to cut one of the bone ends to allow the bone segment replacing the mandible body to be positioned relative to the other bone segment with an inward angle if necessary. The donor bone segments are joined to each other or to existing bones in the patient in known manner.

Alternatively described, the invention is a method of mandible reconstruction comprising the steps of providing a substantially linear donor bone; identifying a portion of a mandible to be reconstructed, said mandible portion being curved or angled; determining an angle between two substantially linear bone segments to replicate the configuration of said mandible portion to be reconstructed; providing a wedge-shaped guide member having two edges joined at a distal end to define an angle corresponding to the difference between the determined angle and 180 degrees; utilizing said guide member to provide a proper cutting angle on said donor bone; cutting said donor bone to produce two bone segments, each having an angled end; and joining together said angled ends of said donor bone segments; whereby upon cutting said donor bone on the angles presented by the guide member and joining said angled ends of said donor bone segment, a construct having a configuration corresponding to said mandible portion is created.

The invention may further comprise providing a plurality of wedge-shaped guide members to create a set, each of said guide members having a different angle at their distal ends.

The invention may further comprise identifying a second portion of said mandible to be reconstructed, said mandible portion being curved or angled; providing a third linear bone segment having an end; determining a second angle between said third linear bone segment of said donor bone and one of said two bone segments to replicate the configuration of said second mandible portion to be reconstructed; choosing a second guide member, said second guide member having two edges joined at a distal end defining an angle corresponding to the difference between said second determined angle and 180 degrees; utilizing said second guide member to provide a proper cutting angle on either said third bone segment or one of said two bone segments; cutting said third bone segment or one of said two bone segments; and joining said third bone segment to one of said two bone segments; whereby upon joining said third bone segment to one of said two donor bone segments, a construct having a configuration corresponding to said second mandible portion is created.

The invention may further comprise a method wherein said step of utilizing said guide member to provide a proper cutting angle on said donor bone comprises marking said donor bone.

The invention may further comprise a method wherein said step of utilizing said guide member to provide a proper cutting angle on said donor bone comprises using said done bone as a cutting guide during the cutting operation.

The invention may further comprise a method wherein said step of utilizing said guide member to provide a proper cutting angle on said donor bone comprises clipping said guide member onto said donor bone.

### BRIEF DESCRIPTION OF THE DRAWINGS

FIG. 1 is a view of a mandible, showing measurement of the main angle between the ramus and the mandible body.

FIG. 2 illustrates a representative donor bone marked with the desired angle for cutting and removing a wedge-shaped portion.

FIG. 3 is a side view representation of the reconstructed donor bone segments joined to form the measured angle of FIG. 1.

FIG. 4 is a top view representation showing the reconstructed donor bone of FIG. 3, the donor bone segments having been reconstructed with an inward turn.

FIG. 5 is a top view representation showing a full reconstructed mandible.

FIG. 6 is a top view representation showing a half reconstructed mandible, the mandible body being comprised of two donor bone segments.

FIG. 7 is a representative embodiment of the guide member having an elongated handle.

FIG. 8 is a representative embodiment of the guide member having a mounting aperture.

FIG. 9 is a representative embodiment of the guide member having a mounting clip.

### DETAILED DESCRIPTION OF THE INVENTION

With reference to the drawings, the invention will now be described in detail with regard for the best mode and the preferred embodiment(s). In a broad sense, the invention is a method, system and set of devices for mandibular reconstruction, the method, system and set of devices utilizing and comprising a plurality of individual wedge-shaped guide members for cutting accurate angles on the ends of donor bone segments such that when the bone segments are joined they will substantially replicate the angled or curved configuration of the mandible or mandible portion being reconstructed.

In circumstances where part or all of mandible needs to be replaced, due for example to injury, cancer, etc., a known technique it to reconstruct the mandible from a donor bone, typically a fibula, by cutting the donor bone into segments and then joining the segments to approximate the shape of the original mandible. Because the mandible is not linear, the ends of the bone segments must be angled to create a corresponding configuration. Typically surgeons perform the reconstruction process by eye, making an estimate as to the proper angle, then shaving or grinding the ends of the cut segment to produce the correct angle.

The vast majority of mandibles have similar angles between the ascending ramus 21, mandibular body 22, and parasymthysis 23. This allows for a system, method and devices that can be considered relatively universal in application, or at least suitable for use in the majority of reconstructive cases, wherein a relatively small number of guide members 10 need to be provided. Alternatively, the method, system and set of devices will include a relatively large number of guide members 10 to allow the surgeon greater latitude in reconstructing the mandible or mandible portion.

A plurality of guide members 10 are provided as a set, each guide member 10 being a wedge-shaped member comprising two edges 15 joined at the distal end 16 of the guide member 10 to define a cutting angle, each guide member 10 in the set having a different angle on the distal end 16. The guide members 10 are preferably produced from a metal, ceramic or similarly hard substance, whereby the surgeon may utilize the guide members 10 either as a template to inscribe or mark the correct cutting angle onto a donor bone 99 or as a directly abutting guide for the blade of the cutting implement, provided the guide members 10 are constructed of suitably durable material. The guide members 10 may be disposable or sterilizable for re-use. The guide members 10 may be provided with gripping members, such as a handle 11, proximal tab 13 or the like, as shown in FIGS. 7 and 8. The guide members 10 may comprise an aperture 12 to be retained on a chain or ring, as shown in FIG. 8. The guide members 10 may comprise a clip 14 for encircling the donor bone 99, as shown in FIG. 9, such that the guide member 10 will be independently retained on the donor bone 99. The thickness of the guide members 10 may vary and the guide members 10 may be curved slightly to better accommodate the curved surface of the donor bone 99.

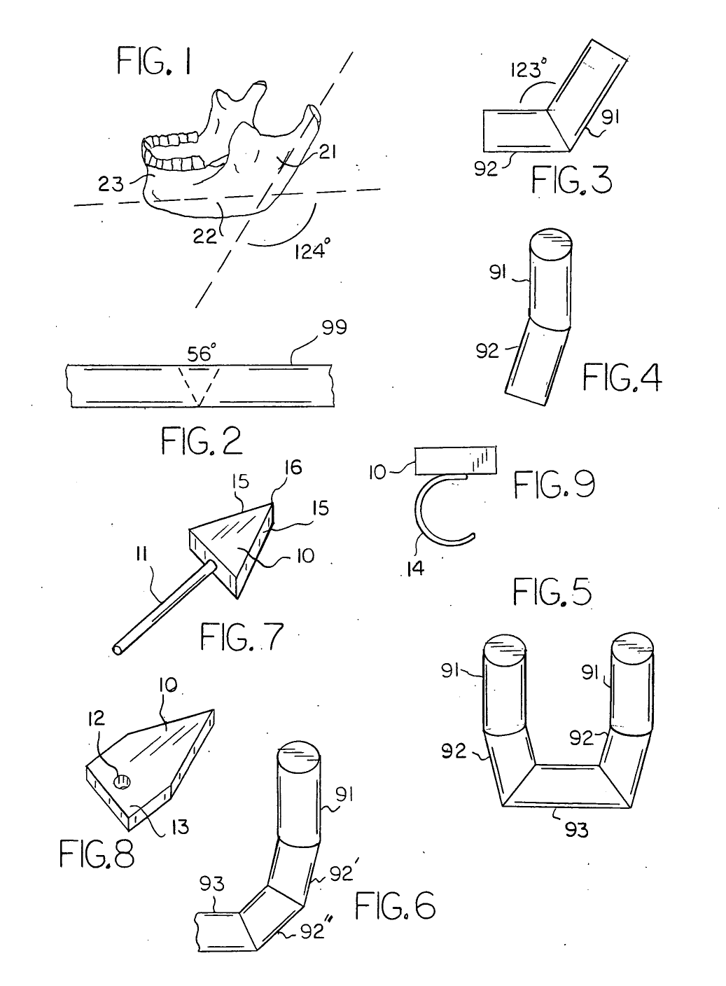

To reconstruct the posterior portion of the mandible, the main angle between the ramus 21 and the mandible body 22 is determined, as shown in FIG. 1. This angle in many circumstances will be approximately 124 degrees. In this circumstance, a guide member 10 having a wedge angle of 56 degrees is chosen to reproduce the angle between the ascending ramus and mandibular body of approximately 124 degrees, as 56 degrees is the difference between the main angle and 180 degrees. The donor bone 99 is cut into two segments 91 and 92 with angled ends based on the inscribed angle of 56 degrees, as shown in FIG. 2. Alternatively, the guide member 10 is used as the cutting guide or template during the actual cutting operation. The angled ends of the two donor bone segments 91 and 92 when joined will create an angle of approximately 124 degrees, replicating the main angle of the ramus 21 and mandible body 22 as shown in FIG. 3. In most circumstances a second angle must be cut or shaved into one or both of the joint ends of bone segments 91 and 92 to produce a proper inwardly angled configuration as shown in FIG. 4. This is accomplished by choosing a guide member of differing angle. For example, if it is determined that the mandible body bone segment should angle inwardly from the original plane of the ramus bone segment by 12 degrees, then a 12 degree guide member is chosen and the end of one of the bone segments 91 or 92 is cut or shaved to produce the desired angle.

In like fashion, if for example the parasymthysis 23 and mandible body 22 need to be reconstructed and the angle between them is approximately 130 degrees, a guide member 10 with an angle of 50 degrees is chosen to provide the proper angle for cutting the linear donor bone 99, after which the two bone segments 92 and 93 are joined in known manner. In the event the mandible body 22 is highly curved, it may be desirable to reconstruct the mandible body 22 using two segments 92′ and 92″, as shown in FIG. 6. As before, this is accomplished by choosing the proper guide member 10 to control the accuracy of the cutting.

It may be possible to reconstruct an entire mandible with only 2 or 3 guide members 10, since the majority of mandibles have similar configurations. To provide better adaptability, a larger number of guide members 10 would be provided as part of the instrumentation set, with for example the guide members 10 in the set varying by for example 5 degrees or 2.5 degrees. With this system the surgeon has more options for correctly reproducing the mandible.

It is understood that equivalents and substitutions for certain elements and steps set forth above may be obvious to those skilled in the art, and therefore the true scope and definition of the invention is to be as set forth in the following claims, and furthermore it is to be understood that the embodiments, examples and illustrations set forth above are not meant to be limiting.

I claim:

1. A method of mandible reconstruction comprising the steps of:
providing a substantially linear donor bone;
identifying a portion of a mandible to be reconstructed, said mandible portion being curved or angled;
determining an angle between two substantially linear bone segments to replicate the configuration of said mandible portion to be reconstructed;
providing a wedge-shaped guide member having two edges joined at a distal end to define an angle corresponding to the difference between the determined angle and 180 degrees;
utilizing said guide member to provide a proper cutting angle on said donor bone;
cutting said donor bone to produce two bone segments, each having an angled end;
and joining together said angled ends of said donor bone segments;
whereby upon cutting said donor bone on the angles presented by the guide member and joining said angled ends of said donor bone segment, a construct having a configuration corresponding to said mandible portion is created.

2. The method of claim 1, further comprising the step of:
providing a plurality of wedge-shaped guide members to create a set, each of said guide members having a different angle at their distal ends.

3. The method of claim 2, further comprising the steps of:
identifying a second portion of said mandible to be reconstructed, said mandible portion being curved or angled;
providing a third linear bone segment having an end;
determining a second angle between said third linear bone segment of said donor bone and one of said two bone segments to replicate the configuration of said second mandible portion to be reconstructed;
choosing a second guide member, said second guide member having two edges joined at a distal end defining an angle corresponding to the difference between said second determined angle and 180 degrees;
utilizing said second guide member to provide a proper cutting angle on either said third bone segment or one of said two bone segments;
cutting said third bone segment or one of said two bone segments;
and joining said third bone segment to one of said two bone segments;
whereby upon joining said third bone segment to one of said two donor bone segments, a construct having a configuration corresponding to said second mandible portion is created.

4. The method of claim 1, wherein said step of utilizing said guide member to provide a proper cutting angle on said donor bone comprises marking said donor bone.

5. The method of claim 1, wherein said step of utilizing said guide member to provide a proper cutting angle on said donor bone comprises using said done bone as a cutting guide during the cutting operation.

6. The method of claim 1, wherein said step of utilizing said guide member to provide a proper cutting angle on said donor bone comprises clipping said guide member onto said donor bone.

7. A method of creating a construct for mandible reconstruction comprising the steps of:
providing a substantially linear donor bone;
identifying a mandible portion to be reconstructed;
determining an angle between two substantially linear bone segments needed to approximate the configuration of said mandible portion to be reconstructed;
providing a wedge-shaped guide member having two edges joined at a distal end to define an angle corresponding to the difference between the determined angle and 180 degrees;
utilizing said guide member to designate a proper cutting angle on said donor bone;
cutting said donor bone to produce two bone segments, each having an angled end;
and joining together said angled ends of said donor bone segments to create a construct approximating the configuration of said mandible portion.

8. The method of claim 7, further comprising the step of:
providing a plurality of wedge-shaped guide members to create a set, each of said guide members having a different angle at their distal ends.

9. The method of claim 8, further comprising the steps of:
identifying a second mandible portion to be reconstructed;
providing a third linear bone segment having an end;
determining a second angle between said third linear bone segment of said donor bone and one of said two bone segments needed to approximate the configuration of said second mandible portion to be reconstructed;
choosing a second guide member, said second guide member having two edges joined at a distal end defining an angle corresponding to the difference between said second determined angle and 180 degrees;
utilizing said second guide member to provide a proper cutting angle on either said third bone segment or one of said two bone segments;
cutting said third bone segment or one of said two bone segments;
and joining said third bone segment to one of said two bone segments;
whereby upon joining said third bone segment to one of said two donor bone segments, a construct having a configuration corresponding to both said mandible portions is created.

10. The method of claim 7, wherein said step of utilizing said guide member to provide a proper cutting angle on said donor bone comprises marking said donor bone.

11. The method of claim 7, wherein said step of utilizing said guide member to provide a proper cutting angle on said donor bone comprises using said done bone as a cutting guide during the cutting operation.

12. The method of claim 7, wherein said step of utilizing said guide member to provide a proper cutting angle on said donor bone comprises clipping said guide member onto said donor bone.
